(19) United States
(12) Patent Application Publication
(10) Pub. No.: US 2017/0296614 A1
(43) Pub. Date: Oct. 19, 2017

(54) Herbal Extract Composition

(71) Applicant: Koppla Ltd., DBA: Koppla Nutrients, New York, NY (US)

(72) Inventors: Ron Sosenko, New York, NY (US); Donna Mascaro, West Windsor, NJ (US); Phil Parisi, Flourtown, PA (US); George Ennis, Newton, PA (US)

(21) Appl. No.: 15/402,418

(22) Filed: Jan. 10, 2017

Related U.S. Application Data
(63) Continuation of application No. 13/750,063, filed on Jan. 25, 2013, now abandoned.

(51) Int. Cl. A61K 36/899; A23L 2/385; A23L 33/105; A61K 36/185; A61K 47/46; A61K 36/53; A23L 2/52

(52) U.S. Cl. CPC A61K 36/899; A61K 36/53; A23L 2/385; A23L 2/52; A61K 36/185; A61K 47/46; A23L 33/105; A23V 2002/00; A61K 2236/17; A61K 2236/37; A61K 2236/53

(57) ABSTRACT

The present invention relates to the field of herbal compositions. In particular, the present invention is directed to an Improved Herbal Extract Composition comprising a Melissa species extract, an Avena species extract, a Tilia species extract and a unique blend of citrus components. The composition can further comprise at least one additional extract of a plant such as Citrus species, Crataegus species, Panax species, and Lavendula species. The herbal extract composition is useful in numerous ingestible forms including an extract concentrate for food and beverage preparation, additive or enhancer for existing foods and beverages and particularly in the form of a pleasant tasting and soothing beverage.

This application is a continuation-in-part of application Ser. No. 13/750,063, filed Jan. 25, 2013 which is a continuation of application Ser. No. 12/625,939, filed Nov. 25, 2009 and incorporated herein in its entirety.

### FIELD OF THE INVENTION

The present invention relates to the field of herbal compositions. In particular, the present invention is directed to an Improved Herbal Extract Composition comprising a Melissa species extract, an Avena species extract, a Tilia species extract, a unique blend of citrus components, and, optionally, at least one additional extract of a plant such as Citrus species, Crataegus species, Panax species, or Lavendula species.

### BACKGROUND TO THE INVENTION

Herbs and spices have long been used in food and beverage preparations. They have been used to enhance flavoring and provide for the user's overall sense of well-being, For example, herbal extract compositions have been described, yet they left an undesirable flavor such as a sour, bitter or earthy taste (see U.S. Pat. No. 5,064,675), Panax ginseng (Asiatic ginseng) provides root-derived preparations that are reported to variously stimulate and relax the nerves (see U.S. Pat. No. 4,446,130 discussed below). Typical dosages recommended for the elderly or for long-term disabilities are 400-800 mg. of dried root equivalent per day. Authorities on the subject recommend for stressful situations, in the young, 600-2,000 mg. of dried root equivalent per day for up to three weeks in any month.

Linden or limeflowers (Tilia europaea) provide flower-derived preparations that are reported to provide a relaxant and healing effect on blood vessel walls. They have been suggested for persons with cardivascular conditions who exhibit pronounced anxiety or tension factor. Dosages of 1-4 g. dried flowers equivalent, three or more times daily have been recommended. Teas prepared from Tilia are recognized as traditional domestic favorites.

Oats (Avena sativa) as whole plant preparations are widely recommended as being generally beneficial for all states of debility, particularly involving the nervous system (see U.S. Pat. No. 4,886,665 discussed below). They have been recommended to be taken for conditions such as depression, and a diverse spectrum of physical diseases. Recommended amounts are 1-4 g. dried herb or oatmeal equivalent three times daily.

Balm mint (Melissa officinalis) is known for its pleasant and characteristic scent. Whole plant preparations are recommended as relaxants for calming the nerves and for improving digestion. It is prepared as a drink to relieve a range of dyspeptic conditions. Recommended amounts are 1-4 g. dried herb equivalent at least three times daily.

Lavender (Lavendula officinalis and other L. species) is recommended for the same type applications as Balm mint. Recommended amounts are 0.5-2 g. dried flowers equivalent three times daily.

Several patents have issued that relate to various uses of herbs and herbal compositions, including the following. Grollier, et al., U.S. Pat. No. 4,767,618, describe cosmetic compositions that use both Melissa and Tilia. Hachiya et al., U.S. Pat. No. 4,446,130, describe a method of preparing dried ginseng and en alcoholic (ethanolic) beverage or elixir and reports that ginseng exhibits sedative, stimulative and diuretic activities.

Kovacs, U.S. Pat. No. 4,886,665, describes a food supplement composition containing an oat extract and an extract of nettle. The combined extracts can be in the form of powder added to a beverage or fruit juice to provide a nutritional drink. The powder products cart be incorporated into other forms.

In contrast, the present invention neutralize the undesirable flavor aspects of The Herbal Extract Composition (U.S. Pat. No. 5,064,675), whether in liquid or dry form, for the purpose of allowing significant therapeutic dose levels of the Herbal Extract to be used in creating commercially viable (high palatability) finished products in the mediums of beverages, foods, supplements, and other ingestible forms.

### SUMMARY OF THE INVENTION

This invention relates to an Improved Herbal Extract Composition that includes a Melissa species extract, an Avena species extract, a Tilia species extract, and a unique blend of citrus components

Desirably, the Herbal Extract Composition includes about 25 to about 75 volume percent of Melissa species extract, about 15 to about 50 volume percent of Avena species extract, about 3 to about 25 volume percent of a Tilia species extract, and about 10 to 50 volume percent of a unique blend of citrus components, depending upon whether the application is dry or liquid.

Preferably, the Herbal Extract Composition comprises about 30 to 65 volume percent of the Melissa species extract, about 20 to about 40 of the Avena species extract, about 5 to about 20 volume percent of the Tilia species extract, and about 15 to about 45 volume percent of the unique blend of citrus components. As with herbal compositions generally, the Improved Herbal Extract Composition of the invention may be beneficially used to provide flavoring and contribute to a user's overall sense of well-being.

The invention also relates to an Improved Herbal Extract Composition encompassing additional extracts such as Citrus, Crataegus, Panax and Lavendula species. The Improved Herbal Extract Composition may take a number of forms such as an extract concentrate that is useful in food and beverage preparations, as an additive or enhancer for solid and liquid foods and beverages. When prepared in water and carbonated water, the Improved Herbal Extract Composition alone can be used to create a pleasant tasting and soothing beverage.

The Improved Herbal Extract Composition may yield the benefits even when the final concentration is substantially lowered by dilution. The enhanced taste, making taste more pleasant and appealing, the benefits realized from foods and beverages, that are made of or contain the Improved Herbal Extract Composition, can include (a) a soothing or calming effect and (b) restoration of focus, sustained energy and activity.

Where Crataegus species, e.g, Crataegus oxyacania, is included in a beverage containing the Improved Herbal Extract Composition, long term restorative effects and enhancement of endurance are realized. Addition of lemon, orange, vanilla, ginger, berry, mango, mint, chocolate, and other such flavor extracts, to the Improved Herbal Extract Composition enhances the pleasant taste of foods and beverages.

Although the beneficial properties realized are a result of the combination of certain essential components of the Improved Herbal Extract Composition, the primary effectors of certain of these properties, even when enhanced by modification by inclusion of other constituents, can be identified, For example, the soothing or calming effect is primarily associated with the presence of balm (Melissa officinalis) and oat (Avena sativa). The restoration of energy and activity is primarily associated with the balm constituent.

One embodiment of the invention is a beverage comprising an Improved Herbal Extract Composition specifically made as a beverage concentrate that is dispersed/dissolved in an aqueous carrier. The beverage may comprise about 1 to about 50 volume percent of an Improved Herbal Extract Composition specifically developed for use as a beverage concentrate. Such a beverage concentrate is formulated for the express purpose of providing the benefits of the improved Herbal Extract Composition. A more desirable beverage composition comprises about 2 to about 45 volume percent of the Improved Herbal Extract Composition in the form of the beverage concentrate, preferably about 5 to about 40 volume percent of the Improved Herbal Extract Composition (in the form of the beverage concentrate), more preferably about 8 to about 35 volume percent of the Improved Herbal Extract Composition (in the form of the beverage concentrate). A most preferred beverage composition contains about 10 to about 30 volume percent of the Improved Herbal Extract Composition (in the form of the beverage concentrate).

Another aspect of the invention is a process for preparing the Improved Herbal Extract Composition. The process includes the steps of:

(a) extracting dried leaves of a Melissa species with heat in a non-toxic extraction solvent such as aqueous ethanol and recovering by filtration the organic residue extract thereof;

(b) extracting an Avena species in powder form in a non-toxic extraction solvent such as aqueous ethanol and recovering by filtration the organic residue extract thereof;

(c) extracting in an ethanol solution the dried flowers of a Tilia species and recovering by filtration the organic residue extract thereof;

(d) preparing a mixture of citrus extracts; and

(e) combining the Melissa, .vena and citrus extracts in the desired proportions.

### DETAILED DESCRIPTION OF THE INVENTION

For simplicity and illustrative purposes, the principles of the present invention are described by referring to various exemplary embodiments thereof, Although the preferred embodiments of the invention are particularly disclosed herein, one of ordinary skill in the art will readily recognize that the same principles are equally applicable to, and can be implemented in other compositions, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments. The terminology used herein is for the purpose of description and not of limitation. Further, although certain methods are described with reference to certain steps that are presented herein in certain order, in many instances, these steps may be performed in any order as would be appreciated by one skilled in the art, and the methods are not limited to the particular arrangement of steps disclosed herein.

The present invention is directed to an improved Herbal Extract Composition comprising a species extract, an Avena species extract, a Tilia species extract and a unique blend of citrus components. The unique blend of citrus components helps to alter/eliminate the undesirable flavor notes that are naturally occurring in previous herbal extract compositions (sour, bitter, earthy, etc.) without imparting a distinct flavor profile to the mix. The composition can further comprise at least one additional extract of a plant such as Citrus species, Crataegus species, Panax species, and Lavendula species. The herbal extract composition is useful in numerous ingestible forms including an extract concentrate for food and beverage preparation, additive or enhancer for existing foods and beverages and particularly in the form of a pleasant tasting and soothing beverage.

The present inventors have surprisingly found that using a citrus mixture as herein provided is a significant improvement, as opposed to a fermented citrus neutralized a specific range of undesirable flavor notes, but imparts a ‘distinct flavor profile’ into all ingestible applications. The fermented citrus did not fully neutralize certain notes that are typical of herbal ingredients, such as: earthy, grassy, woody, etc. Further, because of the “pulpy ” nature of this fermented citrus, it could only be applied to applications utilizing a liquid extract system, it could not be effectively converted to a dry system for dry product applications. The equipment required to produce commercial size volumes of this fermented citrus would have to be custom made, and would be very expensive to construct and the start to finish fermentation process was a minimum of 10 weeks.

The citrus mixture, or DM Capitibilizer, described herein neutralizes the full range of undesirable flavor notes, without imparting any distinct flavor profile that would be carried over to a finished product. This allows for commercial mediums where ‘lemon/citrus’ flavor notes were not desirable in the overall flavor profile: Savory foods such as chips, cereals, soups, plus chocolates, coffee products and tea mixes (tea bags) not wanting to add a lemon character to their already distinctive flavor profiles. DM Capitibilzer comprises any of the following formulations:

DM Capitibilizer formulations

| | | Range |
|---|---|---|
| Citrus Flavor # 1 | use: 0.15% | |
| Lemon oil terpeneless | | 0.10-0.50 |
| Lemon essence | | 0.80-2.4 |
| Lime essence | | 0.10-0.70 |
| Oil of Lemon | | 0.10-0.60 |
| Pine Needle oil | ** | 0.00001-0.00005 |
| ethyl alcohol | | qs |
| Mix instructions: to a clean | | |
| dry container add ethyl alcohol | | |
| and remaining items in order | | |
| and mix well | | |
| Chill sample to 45 F. and filter | | |
| with filter aid. | | |
| Citrus Flavor # 2 | use: 0.15% | |
| Orange oil | | 0.10-0.35 |
| Orange oil Terpenes | | 0.40-1.20 |
| Tangerine oil | | 0.05-0.25 |
| Acetaldehyde | | 0.01-0.045 |
| Ethyl Butyrate | | 0.002-0.008 |
| Benzaldehyde | | 0.001-0.005 |
| Orange essence | | 0.20-0.80 |
| Mandarin oil | | 0.05-0.35 |
| Orange oil concentrate | | 0.15-0.95 |
| citric acid | | 1.0-4.0 |
| water | | 1.5-3.5 |
| Rose oil | ** | .00002-0.001 |
| Ethyl alcohol | | qs |
| Mix instructions: to a clean | | |
| dry container add ethyl alcohol, | | |
| water, citric acid and remaining | | |
| items and mix well | | |
| Chill sample to 45 F. and filter | | |
| with filter aid | | |
| Citrus Flavor # 3 | use: 0.36% | |
| Lemongrass oil | | 0.001-0.0065 |
| Lemon oil | | 0.05-0.40 |
| lemon oil terpenes | | 0.001-0.0075 |
| Lemon peel solid extract | | 0.004-0.012 |
| Lemon Aroma | | 0.005-0.025 |
| lime oil | | 0.005-0.03 |
| lemon juice | | 12-13.5 |
| lemon oil residue | | 0.004-0.012 |
| Nootkatone | | 0.00001-0.0001 |
| Mustard oil | ** | 0.00001-0.0001 |
| Lime oil terpenes | | 0.0005-0.005 |
| Geraniol | | 0.0002-0.001 |
| Neroli oil | | 0.0002-0.001 |
| Propylene Glycol | | 25-30 |
| Water | | 15-20 |
| Ethyl alcohol | | qs |
| Mix instructions: Step 1 to a | | |
| clean dry container add water, | | |
| propylene glycol, lemon juice, | | |
| and solid extract and mix well | | |
| Step 2 in a separate container | | |
| add ethyl alcohol and remaining | | |
| items and mix well | | |
| Step 3 slowly add step 2 to | | |
| step 1 and mix well | | |
| Points of difference: Novelty, | | |
| unique aspect, non obviousness | | |

DM Capitibilizer can be utilized in both liquid and dry extract processes: (DRY): powdered beverage mixes; bulk teas; dissolvable strips, instant soup mixes, etc.

Commercial scale productions of the DM Capitibilizer can be produced with existing equipment, there is ac need to custom make expensive apparatus.

From start to finish, the process to produce commercial size batches of the DM Capitibilizer is less than 2 weeks.

Herbal extracts in accordance with the present invention may be produced as follows:

(a) extract dried leaves of a Melissa species with heat in a non-toxic extraction solvent such as aqueous ethanol and recovering, by filtration the organic residue extract thereof;

(b) extract an Avena species in powder form in a non-toxic extraction solvent such as aqueous ethanol and recovering by filtration the organic residue extract thereof;

(c) extract in an ethanol solution the dried flowers of a Tilia species and recovering by filtration the organic residue extract thereof;

(d) prepare a mixture of citrus extracts; and

(e) combine the Melissa, Avena, Tilia, and citrus extracts in the desired proportions.

Improved Herbal Extract Compositions in accordance with the present invention may comprise about 40 to about 70 volume percent of a citrus extract and about 30 to about 60 volume percent of the herbal extracts.

When present, a Panax extract may comprise about 0.5 to 2 volume percent of the Improved Herbal Extract Composition. When present, a Crataegus extract may comprise about 0.5 to 2 volume percent, the remainder being Improved Herbal Extract Composition.

Beverage in accordance with the invention may comprise a beverage concentrate dissolved/dispersed in an aqueous carrier. The aqueous cattier may be distilled water, spring water, carbonated water, filtered water, and the like. The amount of the Improved Herbal Extract Composition contained in the beverage is dependent on a number of considerations. One consideration is taste and another is the desired physiological effect sought from ingestion of the beverage. The desired concentrations noted above is intended to be variable in recognition of these considerations.

The extraction processes used to isolate the desired extracts used in forming the Herbal Extract Composition and Modified Extract Composition of the invention utilizes traditional techniques in the art. The extraction is typically carried out at a temperature below that which adversely affects the properties of the extracted herbal composition or is above a reasonable reflux temperature for the extraction solvent of choice, and above that at which meaningful extraction does not occur.

Generally, such temperatures range from as low as about 0° C. to 300° C., though temperatures that are higher or lower are feasible. Generally, the temperature of the extraction is dependent upon the boiling or freezing characteristic of the extraction solvent or solution. Moderate temperatures are preferred so long as extraction efficiencies for economic extraction rates are met. As a rule, higher temperatures are reflected in higher extraction rates.

The choice of extraction solvent is typically dependent upon the chemical nature of the herbal component undergoing extraction, the toxicity characteristics of the solvent, boiling and freezing points of the solvent, and the like considerations. Alcohols, such as ethanol, etc., are quite suitable as extraction solvents, neat or dissolved in water, and acetone, diethylether and aqueous ammonia are useful solvents in select cases. Ethanol is the preferred extraction solvent.

As a specific example of extraction for balm mint (Melissa officinalis), the starting material comprises 1 part dried leaves in 5-25 parts aqueous ethanol solution containing 60 weight % ethanol. The leaves are extracted with ethanol at 80°-95° C. and the organic residue is pressed and filtered when the temperature has reached ambient conditions. The extract has a shelf life of at least 3 years.

As a specific example of extraction for oat (Avena sativa), the starting material comprises 1 part herb material in 1-5 parts ethanol (e.g. 100 g plant material per 100-500 g ethanol). For example, the plant is extracted with pure water and ethanol (95%) for 4-10 weeks and stirred on a daily basis. The organic residue is thereafter pressed and filtered. The extract has a shelf life of at least about 3 years.

As a specific example of extraction for linden (Tilia europaea), the starting material comprises 1 part dried flowers per 5-25 parts ethanol (e.g. 100 g flowers per 500-2500 g of 60% ethanol). When the mixture has reached ambient conditions, it is pressed and filtered. The extract has a shelf life of at least about 3 years.

The optionally included Crataegus species extract is prepared by extracting the fruit and leaves in water and then in ethanol and recovering by filtration the organic residue extract thereof. Similarly, the optionally included Panax species extract is prepared by refluxing the dried root in a dilute aqueous ethanol and recovering by filtration the organic residue extract thereof. As well, the optionally included Lavendula species extract is prepared by extracting with heat the dried flowers in ethanol and recovering by filtration the organic residue extract thereof.

As a specific example of extraction for lemon (Citrus medica), the starting material comprises 1 part dried fruit in 2-10 parts water. The lemon juice is recovered and the peels are sliced. The juice and sliced peels are fermented at room temperature for 3-10 weeks and the organic residue is thereafter pressed and filtered. The clear extract is stored at 4° C.

As a specific example of extraction for Crateaegus (Crataegus oxycanta), the starting material comprises fruit and leaves (5:1 to 1:1 ratio w/w). The organic material is extracted in water (e.g. at 37° C.) for 0.5-3 hours. Thereafter, ethanol (95%) is added to reach a final ethanol concentration in the range of about 10-50%. The mixture is left for 4-12 weeks at 5°-30° C. After this period, the residue is pressed and filtered. The extract has a shelf life of at least about 3 years.

As a specific example for Panax (Panax ginseng), the starting material comprises 1 part dried ginseng root material per 2-20 parts ethanol. Organic material is refluxed for at least about 30 minutes in ethanol (10-50%). The residue is pressed and filtered when it has reached ambient temperature, The extract has a shelf life of at least about 3 years.

As a specific example for lavender (Lavendula officinalis), the starting material comprises 1 part dried lavender flower material per 10 parts ethanol Flowers are extracted with 60% ethanol (80°-95° C.). The organic residue is pressed and filtered when the temperature has reached ambient. The extract has a shelf life of at least about 3 years.

One preferred embodiment provides a non-alcoholic beverage of particularly preferred extract proportions, e.g., a balm mint leaf extract, an oat plant extract and a linden flower extract, Preferably, the Melissa species extract comprises about 40 to 60 volume percent, the Avena species extract comprises about 20 to 30 volume percent and the remainder was Tilia species. An exemplary formulation of the beverage of the invention is DM Capitibilizer (27.00 ml), Melissa officinalis extract (19.00 ml), Avena sativa extract (9.75 ml), Tilia europaea extract (4.50 ml), Panax ginseng extract (0.65 ml), Crataegus oxyacanta extract (0.65 mi) and Lavendula officinalis extract (0.07 ml), a total of 61.62 ml of extract, in 2,000 ml of natural spring water. Optionally, sugar can be included.

### EXAMPLE 1

The step-by-step preparation and process of formula for the mixture of citrus extracts, referred to herein as “DM Capitibilizer” is as follows:

Ingredients

The DM Capitibilzer formula may be composed of a complex mixture of extracts, including: Lime, Lemon, Mandarin, Orange, Tangerine and Grapefruit. Along with these, there are additions of both expressed and distilled citrus oils, and specific components isolated from citrus, such as neroli and geraniol. These are natural aroma isolates which enhance citrus, and give this composition its unique neutral signature flavor character.

When this flavor character is used in conjunction with the botanical herbs in the Koppla blend, the finished blend that is elicited becomes the stepping stone for creating commercially viable ingestible products where efficacious dose levels of the medicinal herbs can be utilized, with all flavor related barriers of the herbal blend being neutralized, thus enabling outstanding tasting finished products.

Prepping the Ingredients Prior to Mixing

The oils of the lemon, lime, mandarin, orange, tangerine and grapefruit are extracted from the fresh fruit peel by the process of cold pressing or expressing, separating the oil from the flavedo in the peel.

Folding is a concentration process used during which a certain percentage of terpenes are removed from the starting oil by distillation. When a percentage of terpenes are removed, the percentage of other compounds increase.

Re-distillation of these essential oils can remove terpenes to make the oil more hydro-alcoholic soluble.

The deterpenation renders the oils terpeneless.

A fluid extract of the peels from each of the 6 citrus fruits used, along with alcohol and water, are macerated for ten days in a closed vessel with occasional agitation.

The mixture is then pressed, filtered, and the solvent removed, resulting in a solid extract from EACH of the 6 citrus peels.

Blending Instructions and Equipment

Step 1: To a clean sanitized lightning style mixing tank, add ethyl alcohol and remaining ingredients in order of addition by weight, lowest to highest.

Step 2: Mix well for 30 minutes, or until a homogeneous mixture is achieved.

Step 3: Chill the DM Capitibilzer to between 45F to 40F.

Step 4: Filter until clear using diamateceous filter aid.

Step 5: Sample to quality assurance for analysis and approval

Effects of Individual Components Upon Neutralizing Undesirable Flavor Notes

Sinensal, a mixture of alpha and beta isomers: This sesquiterpene aldehyde from orange, tangerine, and mandarin, helps neutralize bitter and sour aspects of the botanical notes of the herbal blend.

The terpene valencene and precursor to nootkatone, a significant component of grapefruit, helps neutralize aspects of the grassy notes of the herb blend.

Saturated aldehydes of decenal nonanal, and octanal from terpeneless orange, lemon and lime oils, neutralize aspects of the earthy notes of the herb blend.

The saturated open chain esters of decyl, nonyl, octyl, and heptyl acetate, along with the unsaturated esters of neryl, geranyl, citronelly and terpinyl acetates helps to neutralize aspects of the oat grain fermented notes of the herb blend.

The alcohols and esters of cis-3-hexenol, nerol, geraniol and citronellol combined with ethyl and hexyl butyrate, decyl and hexyl acetate helps neutralize aspects of the medicinal notes of the herbal blend.

The expressed citrus oil terpenes dextro limonene, alpha and beta pinene, sabinene, gamma terpinene and terpinolene help neutralize aspects of the resinous bark notes of the herbal blend.

Composition of Citruses (6) used in one example of DM Capitibilizer blend (‘percentage use range’ of each citrus extract)

Lemon extract comprises: 80.0-92.0% of the 6 citrus fruits blend

Orange extract comprises: 5.0-11.0% of the 6 citrus fruits blend

Lime extract comprises: 1.2-2.2% of the 6 citrus fruits blend

Mandarin extract comprises: 0.4-0.8% of the 6 citrus fruits blend

Tangerine extract comprises: 0.4-0.8% of the 6 citrus fruits blend

Grapefruit extract comprises: 0.02-0.04% of the 6 citrus fruits blend

### EXAMPLE 2

Converting the LIQUID DM Capitibilzer to a DRY system (Spray drying)

An emulsion is prepared with DM Capitibilizer, water, and a selection of film forming components such as: gum Arabic, modified food starch, and maltodextrins, also referred to as the carrier system.

The emulsion is formulated to a precise emulsion solids to water ratio, determined by the carrier used.

Then the mixture is homogenized and optimized to an established lipid particle size distribution and viscosity, determined by the carrier used.

The DM Capitibilizer emulsion is fed into a surge tank and then metered into the drying chamber of a spray dryer that has been preheated, and has a preheated hot air stream set a predetermined temperature, within the 160-220C range.

The DM Capitibilizer emulsion droplets are dispersed by a centrifugal head atomizer or nozzle atomizer into the heated air stream within the plenum of the dryer at a specific rate,

Each liquid droplet rapidly gives up moisture at the air-liquid interface forming a film on the outer droplet surface.

Water molecules rapidly diffuse out of the droplet increasing the solids content of the particle as it dries.

The DM Capitibilzer becomes entrapped within the matrix of the carrier system.

A sample of the spray dried DM Capitibilizer is collected and passed to quality control for analytical testing and approval.

### EXAMPLE 3

Blend ratios of DM Capitibilizer to Koppla Herbal Blend=Finished enhanced Herb blend

A LIQUID version of the DM Capitibilizer is comprised of:

6-12% citrus juice components

8-12% citrus extractives

6-8% water

15-20% carrier

56-65% grain alcohol

The preferred application of the completed liquid DM Capitibilizer is at a ratio of 43.92% to 56.08% of the liquid Koppla herbal blend, creating the desired neutralizing effect upon the undesirable flavor notes inherent with the herbal blend.

A SPRAY DRY version of the DM Capitibilizer may be comprised of:

1-3% citrus juice components

12-19% citrus extractives

8-87% carrier

The preferred application of the completed Spray Dry DM Capitibilizer is at a ratio of 16.12% to 83.88% of the dry Koppla herbal blend, creating the desired neutralizing effect upon the undesirable flavor notes inherent within the herbal blend.

### EXAMPLE 4

Beverage Evaluation Procedure:

In support of the Koppla Improved Herbal Extract patent, this evaluation provided expert flavor guidance on the commercial viability of several Koppla RID beverages. By conducting this tasting, we wanted to demonstrate that the combination of the DM Capitibilizer and distinct flavors (vanilla, ginger, mango and cherry) creates commercially viable flavor profiles. We also planned to show that the addition of the DM Capitibilizer alone without a flavor (Sample 1B) provides a profile that is not commercially viable. Lastly, we wanted to determine that the exclusion of the DM Capitibilizer with a flavor yields a profile that is not commercially viable. We conducted a simple taste evaluation using five David Michael flavor experts as panelists. Sample evaluations were conducted at a conference table in the David Michael Sensory Lab. The sensory lab is a controlled environment, maintaining a constant temperature and allowing minimal noise interference.

Panelists were served samples of each product in labeled 1 oz. cups. Samples were evaluated at room temperature and were served in pairs, based on flavor. The sample not containing the DM Capitibilizer was always evaluated as the first sample of each pair. A score sheet was provided as a guide. Panelists evaluated the pairs by writing down descriptor terms based on aroma and flavor for each. This was done individually at first using the score sheet, followed by a group discussion.

During the group discussion, each panelist verbally expressed his/her description (list of terms) to the rest of the group. This was done for each sample of the current pair. The terms were written by the discussion leader on an easel board. As the list of terms grew, similar flavor descriptors were discussed to form a consensus of terms used to describe each sample. Although different descriptor terms were used to describe a characteristic of a sample, the meaning of the terms used for that characteristic was agreed to be the same by the panelists, so one descriptor term was agreed upon. The reason for forming a consensus versus listing all attributes named is to reduce redundant terms that have the same meaning. Once a description was created for a sample, the group determined if the sample was “commercially viable” or “not commercially viable.”

Overall Findings from the Evaluations:

The addition of DM Capitibilizer without a flavor (Sample 1B) did not provide a commercially viable flavor profile, although some of the bitter and metallic notes were covered by DM Capitibilizer in this product that were not covered by the sample without the DM Capitibilizer (Sample 1A).

The combination of DM Capitibilizer and distinct flavors provided commercially viable flavor profiles (Samples 2B, 3B, 4B and 5B),

When DM Capitibilizer was excluded from a flavored product, we confirmed those products were not commercially viable. The only exception being mango, for which both Samples 4A and 4B are both commercially viable. The combination of mango and DM Capitibilizer (Sample 4B) provides an excellent base on which to add another flavor, to differentiate it from Sample 4A, The same is true for ginger. Although the combination of DM Capitibilizer and ginger (Sample 3B) was a marked improvement over Sample 3A, ginger tends to be polarizing with consumers. Pairing ginger with another flavor will create a unique profile for consumers.

A flavor profile summary of each flavor pair is shown in Tables 4-8.

TABLE 4

| Control A | Control B |
|---|---|
| 4A-No DM Capitibilizer, | 4B-Added DM Capitibilizer, |
| No Added | No Added |
| Flavoring: NOT Commercially | Flavoring: NOT Commercially |
| Viable | Viable |
| Aroma: | Aroma: |
| Herbaceous, grassy | Citrus (lemon/lime) |
| Fruit Cocktail Syrup | Herbaceous, grassy |
| | Fruity |
| Flavor: | Flavor: |
| Herbaceous, botanical | Herbaceous, botanical |
| Earthy | Earthy |
| Grassy/Green | Grassy |
| Fruity (skin/pulp) | Bitter (lower intensity compared to 1A) |
| Metallic, bitter | Lemon, lime, peely |
| Sour | |

General Comments:
The samples have the same general flavor characteristics. The bitter and
metallic notes present in Sample 1A are not as intense in Sample 1B.

TABLE 5

| Vanilla A | Vanilla B |
|---|---|
| 5A-No DM Capitibilizer, + | 5B-Added DM Capitibilizer, |
| Vanilla Flavor | Vanilla Flavor: |
| NOT Commercially Viable | Commercially Viable |
| Aroma: | Aroma: |
| Vanillic | Vanillic |
| Earthy, grassy | Fruity |
| Fruity | Earthy notes diminished (masked, |
| Sour | covered) |
| Herbaceous | Cola spices (brown spice) |
| Flavor: | Flavor: |
| Unbalanced | Citrus |
| Medicinal, dirty | Vanilla |
| Bark, resinous | Earthy notes diminished (masked, |
| Herbaceous | covered) |
| | Balanced |
| | Lemon, lime (lemon/lime soda notes) |
| | Sweetness perception enhanced |

General Comments:
Sample 2B is well balanced and the addition of DM Capitibilizer
masks the medicinal off notes present in Sample 2A.

TABLE 6

| Ginger A | Ginger B |
|---|---|
| 6A-No DM Capitibilizer, + | 6B-Added DM Capitibilizer, + |
| Ginger Flavor | Ginger |
| NOT Commercially Viable | Flavor: Commercially Viable |
| Aroma: | Aroma: |
| Ginger | Citrus |
| Piney | Ginger (ginger ale) |
| Earthy | |
| Grassy | |
| Flavor: | Flavor: |
| Piney (Christmas tree-like) | Earthy, woody notes masked, covered |
| Earthy, woody | Ginger ale |
| Grassy | Citrus |
| Spice (ginger) | Clean, no lingering off-noted |
| Heat (from ginger) linger | Heat (from ginger) - lingers |
| Perfumy | Refreshing, balanced flavors |
| Unbalanced flavors, notes linger | |

General Comments:
Off notes present in Sample 3A are masked in Sample 3B with the
addition of DM Capitibilizer. Ginger is a polarizing flavor to consumers
so adding a flavor to complement the ginger in 3B will help differentiate
this product further.

TABLE 7

| Mango A | Mango B |
|---|---|
| 7A-No DM Capitibilizer, + | 7B-Added DM Capitibilizer, |
| Mango Flavor | Mango Flavor: |
| Commercially Viable | Commercially Viable |
| Aroma: | Aroma: |
| Tropical, sulfur | Citrus |
| Fruity | Lemon, lime |
| Unripe mango (green) | Herbal |
| | Mango (ripe) |
| Flavor: | Flavor: |
| Tropical, sulfur | Mango (ripe) |
| Mango, citrus | Citrus (lemon, lime) |
| Peach, Papaya | Mandarin orange |
| Earthy | |
| Buccu, thiol | |

General Comments:
Adding DM Capitibilizer to 4B created a ripe mango flavor. Both options
are commercially viable; 4B will be more differentiated by adding a
complementary flavor.

TABLE 8

| Cherry A | Cherry B |
|---|---|
| 8A-No DM Capitibilizer, + | 8B-Added DM Capitibilizer, |
| Cherry Flavor | Cherry Flavor |
| NOT Commercially Viable | Commercially Viable |
| Aroma: | Aroma: |
| Candy | Candy |
| Cherry, Kool-Aid, benzaldehyde | Fruit punch |
| | Citrus (lemon, lime) |
| Flavor: | Flavor: |
| Herbaceous | Cherry (adult-type) |
| Bitter, medicinal, cough syrup | Fruit punch |
| Cherry, benzaldehyde | Lime |
| Cherry pit, cherry bark | Cherry cola (cherry, spices) |
| *flavor differs from aroma | Off-notes masked, covered (bitter, |
| | medicinal) |

General Comments:
With the addition of DM Capitibilizer, off-notes are covered and
it creates a cherry profile that can be geared towards adults.

1. An Herbal Extract Composition comprising:
a Melissa species extract;
an Avena species extract;
a Tilia species extract; and
an additional composition comprising a citrus mixture and at least one of pine needle oil, rose oil and mustard oil.

2. The Herbal Extract Composition of claim 1 wherein the Improved Herbal Extract Composition comprises about 25 to about 75 volume percent of Melissa species extract, about 15 to about 50 volume percent of Avena species extract, about 3 to about 25 volume percent of a Tilia species extract, and about 20 to about 55 volume percent of the additional composition comprising a citrus mixture and at least one of pine needle oil, rose oil and mustard oil.

3. The Herbal Extract Composition of claim 2 wherein the Herbal Extract Composition comprises about 30 to 65 volume percent of the Melissa species extract, about 20 to about 40 of the Avena species extract; about 5 to about 20 volume percent of the Tilia species extract, and 30 to about 50 volume percent of additional composition comprising a citrus mixture and at least one of pine needle oil, rose oil and mustard oil.

4. An Herbal Extract Composition comprising the Herbal Extract Composition of claim 1 and one or more of other extracts.

5. The Herbal Extract Composition of claim 4 wherein the other extracts include one or more of Citrus, Crataegus, Panax and Lavendula species.

6. The Herbal Extract Composition of claim 5 in the form of an extract concentrate that is useful in food and beverage preparations, as an additive or enhancer for solid and liquid foods and beverages.

7. The Herbal Extract Composition of claim 6 in the form of a beverage concentrate composition.

8. The Herbal Extract Composition of claim 7 wherein the beverage concentrate composition contains a lemon, orange, vanilla, ginger, berry, mango, chocolate, mint, or other such extracts to enhance taste.

9. A beverage comprising the Beverage Concentrate Composition of claim 8 dispersed/dissolved in an aqueous carrier.

10. The beverage of claim 9 containing about 1 to about 50 volume percent of the beverage concentrate composition.

11. The beverage of claim 10 containing about 2 to about 45 volume percent of the beverage concentrate composition.

12. A beverage comprising the beverage concentrate composition of claim 7 dispersed/dissolved in an aqueous carrier.

13. The beverage of claim 12 containing about 1 to about 50 volume percent of the beverage concentrate composition.

14. The beverage of claim 13 containing about 2 to 15 about 45 volume percent of the beverage concentrate composition.

15. The Improved Herbal Extract Composition of claim 4 in the form of an extract concentrate that is useful in food and beverage preparations, as an additive or enhancer for solid and liquid foods and beverages.

16. A process for preparing an Herbal Extract Composition which comprises:
(a) extracting dried leaves of a Melissa species with heat in a non-toxic extraction solvent and recovering by filtration the organic residue extract thereof;
(b) extracting an Avena species in powder form in a non-toxic extraction solvent and recovering by filtration the organic residue extract thereof;
(c) extracting in a non-toxic extraction solvent the dried flowers of a Tilia species and recovering by filtration the organic residue extract thereof;
(d) preparing an additional composition comprising a citrus mixture and at least one of pine needle oil, rose oil and mustard oil; and
(e) combining the Melissa, Avena and Tilia extracts with the additional composition comprising a citrus mixture and at least one of pine needle oil, rose oil, and mustard oil in the desired proportions to form the Herbal Extract Composition.

17. The process of claim 16 wherein the fruit, flowers, and leaves of a Crataegus species in water and then in ethanol is extracted, and recovered by filtration the organic residue extract thereof and thereafter combined in the formation of an Herbal Extract Composition.

18. The process of claim 16 wherein dried root of a Panax species is extracted in a refluxing dilute aqueous ethanol solution and the organic residue extract thereof is recovered by filtration and combined in the formation of an Herbal Extract Composition.

19. The process of claim 16 wherein the dried flowers of a Lavendula species are extracted with heated ethanol and the organic residue extract is recovered by filtration, and combined in the formation of a Improved Herbal Extract Composition.

20. An Herbal Extract Composition comprising:
a Melissa species extract;
an Avena species extract;
a Tilia species extract;
a citrus mixture; and
at least one of pine needle oil, rose oil, and mustard oil.
